(19) United States
(12) Patent Application Publication
(10) Pub. No.: US 2026/0354693 A1
(43) Pub. Date: Nov. 12, 2026

(54) BLOOD PRESSURE MEASUREMENT DEVICE, BLOOD PRESSURE MEASUREMENT METHOD, BLOOD PRESSURE MEASUREMENT RECORDING MEDIUM, AND METHOD FOR GENERATING CLASSIFIER

(71) Applicant: OMRON HEALTHCARE Co., Ltd., Kyoto (JP)

(72) Inventors: Yuki OTA, Kyoto (JP); Daizo OKA, Kyoto (JP); Wataru TSUNODA, Kyoto (JP); Naomi MATSUMURA, Kyoto (JP)

(21) Appl. No.: 19/713,984

(22) Filed: Jun. 19, 2026

Related U.S. Application Data
(63) Continuation of application No. PCT/JP2024/040251, filed on Nov. 13, 2025.

(30) Foreign Application Priority Data
Feb. 27, 2024 (JP) 2024-027772

(51) Int. Cl. A61B 5/021; A61B 5/00

(52) U.S. Cl. CPC A61B 5/02141; A61B 5/6843; A61B 5/7267

(57) ABSTRACT

A blood pressure measurement device including: a cuff to be wrapped around a measurement target site; a pressure detection unit that detects a cuff pressure inside the cuff; a pressure control unit that controls the cuff pressure; a blood pressure calculation unit that performs blood pressure calculation processing of calculating a blood pressure of a subject, based on the detected cuff pressure; and a wrapping strength determination unit that performs wrapping strength determination processing of determining a wrapping strength of the cuff around the measurement target site. The pressure control unit performs constant-rate pressurization control such that the cuff pressure is pressurized at a constant rate through the wrapping strength determination processing performed after a start of pressurization of the cuff pressure and the blood pressure calculation processing performed after an end of the wrapping strength determination processing.

### CROSS-REFERENCE TO RELATED APPLICATIONS

This application is the U.S. national stage application filed pursuant to 35 U.S.C. 365(c) and 120 as a continuation of International Patent Application No. PCT/JP 2024/040251, filed Nov. 13, 2024, which application claims priority to Japanese Patent Application No. 2024-027772, filed Feb. 27, 2024, which applications are incorporated herein by reference in their entireties.

### TECHNICAL FIELD

The present invention relates to a blood pressure measurement device, a blood pressure measurement method, a blood pressure measurement recording medium or program, and a method for generating a classifier.

### BACKGROUND ART

In recent years, it has become widespread to perform health management by measuring information related to a body and health of an individual, such as a blood pressure value, by using a measurement apparatus, and recording and analyzing the measurement result. As an example of the measurement apparatus as described above, a sphygmomanometer is known which has a function of determining a wrapping strength of a cuff worn on a measurement target site such as an upper arm or a wrist of a subject (see Patent Literature 1).

In the sphygmomanometer disclosed in Patent Literature 1, after the cuff is worn, a pump is driven by applying a constant voltage to the pump during initial pressurization, the wrapping strength of the cuff is determined from a cuff pressure-pressurization time characteristic (cuff pressure curve) from an initial pressure to a pressure value at the end of the initial pressurization, and subsequently, blood pressure calculation processing is performed.

However, in this way, the pressurization rate of the cuff pressure changes abruptly at the timing when the control switches from constant voltage drive to constant-rate pressurization control after the wrapping strength is determined, making it difficult to acquire a clean pressure pulse wave.

### CITATION LIST

### Patent Literature

Patent Literature 1: JP 5408142 B

### SUMMARY OF INVENTION

### Technical Problem

In light of the related art described above, an object is to provide a technology that suppresses the generation of noise associated with the switching of control from cuff wrapping strength determination to blood pressure calculation, and enables the acquisition of a clean pressure pulse wave even at low pressure.

### Solution to Problem

In order to solve the above problems, the present invention provides a blood pressure measurement device including:
a cuff configured to be wrapped around a measurement target site;
a pressure detection unit configured to detect a cuff pressure inside the cuff;
a pressure control unit configured to control the cuff pressure;
a blood pressure calculation unit configured to perform blood pressure calculation processing of calculating a blood pressure of a subject, based on the detected cuff pressure; and
a wrapping strength determination unit configured to perform wrapping strength determination processing of determining a wrapping strength of the cuff around the measurement target site,
in which the pressure control unit performs constant-rate pressurization control such that the cuff pressure is pressurized at a constant rate through the wrapping strength determination processing performed after a start of pressurization of the cuff pressure and the blood pressure calculation processing performed after an end of the wrapping strength determination processing.

According to this, constant-rate pressurization control is performed through the wrapping strength determination processing and the subsequent blood pressure calculation processing, thereby suppressing the generation of noise associated with the switching of control and enabling the acquisition of a clean pressure pulse wave even at low pressure during blood pressure measurement.

In the present invention,
the wrapping strength determination unit may determine the wrapping strength in the wrapping strength determination processing, based on time changes in the cuff pressure and in a pressurization rate of the cuff pressure.

In the present invention,
the wrapping strength determination unit may determine the wrapping strength in the wrapping strength determination processing by using a trained classifier that classifies the wrapping strength, based on input time changes in the cuff pressure and in the pressurization rate of the cuff pressure.

In this way, by using a trained classifier, the wrapping strength of the cuff can be determined with greater accuracy.

In the present invention,
the blood pressure measurement device may further include a determination condition judgment unit configured to judge whether a condition for starting the wrapping strength determination processing is satisfied.

According to this, the wrapping strength can be determined in a state where a condition for starting the wrapping strength determination is satisfied, thus enabling more accurate determination.

In the present invention,
the blood pressure measurement device may further include a circumference discrimination unit configured to discriminate a circumference of the measurement target site around which the cuff is to be wrapped.

According to this, it is possible to determine the wrapping strength while also considering the circumference of the measurement target site.

In the present invention,
the blood pressure calculation unit may judge whether to perform the blood pressure calculation processing according to a result of the wrapping strength determination processing.

According to this, the blood pressure calculation processing can be performed only when the wrapping strength of the cuff is appropriate, thus enabling more accurate blood pressure measurement.

In the present invention,
a target value of a pressurization rate of the cuff pressure in the constant-rate pressurization control during the wrapping strength determination processing may be equal to a pressurization rate of the cuff pressure in the constant-rate pressurization control during the blood pressure calculation processing.

According to this, constant-rate pressurization control is performed at the same pressurization rate through the wrapping strength determination processing and the subsequent blood pressure calculation processing, thereby further suppressing the generation of noise associated with the switching of control.

The present invention provides a blood pressure measurement method including:
detecting a cuff pressure inside a cuff configured to be wrapped around a measurement target site;
performing constant-rate pressurization control of maintaining a pressurization rate of the cuff pressure at a constant target value;
performing wrapping strength determination processing of determining a wrapping strength around the measurement target site during the constant-rate pressurization control; and
performing blood pressure calculation processing of calculating a blood pressure of a subject, based on the cuff pressure during the constant-rate pressurization control.

According to this, constant-rate pressurization control is performed through the wrapping strength determination processing and the blood pressure calculation processing, thereby suppressing the generation of noise associated with the switching of control and enabling the acquisition of a clean pressure pulse wave even at low pressure during blood pressure measurement.

The present invention provides a blood pressure measurement program for causing a computer to execute:
detecting a cuff pressure inside a cuff configured to be wrapped around a measurement target site;
performing constant-rate pressurization control of maintaining a pressurization rate of the cuff pressure at a constant target value;
performing wrapping strength determination processing of determining a wrapping strength around the measurement target site during the constant-rate pressurization control; and
performing blood pressure calculation processing of calculating a blood pressure of a subject, based on the cuff pressure during the constant-rate pressurization control.

According to this, constant-rate pressurization control is performed through the wrapping strength determination processing and the blood pressure calculation processing, thereby suppressing the generation of noise associated with the switching of control and enabling the acquisition of a clean pressure pulse wave even at low pressure during blood pressure measurement.

The present invention provides a method for generating a classifier used in wrapping strength determination processing in a blood pressure measurement program for causing a computer to execute:
detecting a cuff pressure inside a cuff configured to be wrapped around a measurement target site;
performing constant-rate pressurization control of maintaining a pressurization rate of the cuff pressure at a constant target value;
performing the wrapping strength determination processing of determining a wrapping strength around the measurement target site during the constant-rate pressurization control; and
performing blood pressure calculation processing of calculating a blood pressure of a subject, based on the cuff pressure during the constant-rate pressurization control, the method including:
machine-learning a classifier configured to output the wrapping strength around the measurement target site as a classification result in response to input of time changes in the cuff pressure and in the pressurization rate of the cuff pressure by using a time change in the cuff pressure, a time change in the pressurization rate of the cuff pressure, and the wrapping strength around the measurement target site as learning data; and
generating the trained classifier.

According to this, it is possible to provide a classification that allows for accurate classification of the wrapping strength of the cuff in blood pressure measurement.

### Advantageous Effects of Invention

According to the present invention, the generation of noise associated with the switching of control from cuff wrapping strength determination to blood pressure calculation is suppressed, and a clean pressure pulse wave can be acquired even at low pressure.

### BRIEF DESCRIPTION OF DRAWINGS

FIG. 1 is a schematic diagram illustrating a hardware configuration of a blood pressure measurement device according to Example 1.

FIG. 2 is a functional block diagram of the blood pressure measurement device according to Example 1.

FIG. 3 is a flowchart illustrating an entire processing procedure of the blood pressure measurement device according to Example 1.

FIG. 4(A) and FIG. 4(B) are graphs illustrating time changes in cuff pressure, pressurization rate, and pulse wave amplitude for subjects with different arm circumferences using the blood pressure measurement device according to Example 1.

FIG. 5 is a flowchart illustrating a procedure for wrapping strength determination processing in the blood pressure measurement device according to Example 1.

FIG. 6 is a graph illustrating a time change in cuff pressure for different wrapping strengths of the blood pressure measurement device according to Example 1.

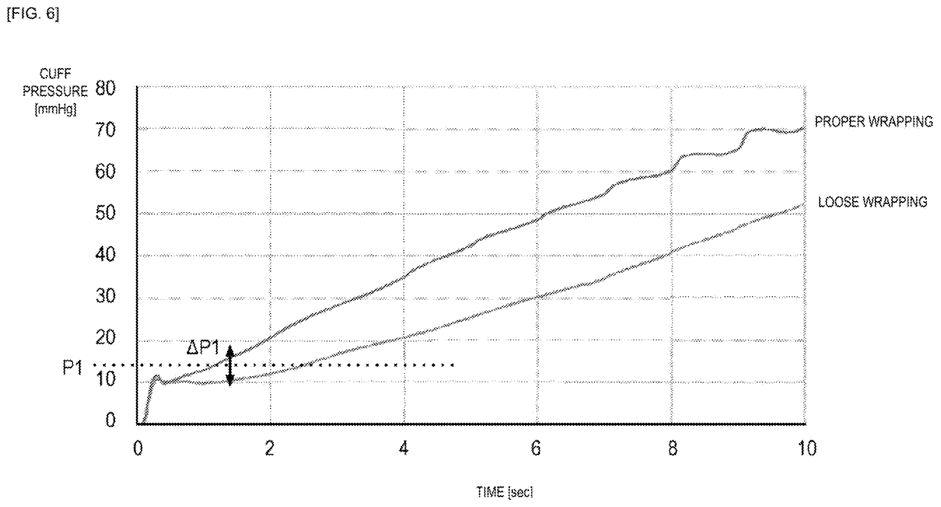

FIG. 7 is a functional block diagram of a blood pressure measurement device according to Example 2.

FIG. 8 is a flowchart illustrating a procedure for arm circumference discrimination and wrapping strength determination processing in the blood pressure measurement device according to Example 2.

FIG. 9 is a graph illustrating a time change in cuff pressure for different arm circumferences and different wrapping strengths of the blood pressure measurement device according to Example 2.

### DESCRIPTION OF EMBODIMENTS

Hereinafter, specific embodiments of the present invention will be described with reference to the drawings.

### Example 1

Hereinafter, an example of the embodiments of the present invention will be described. It should be noted that the dimension, material, shape, relative arrangement, and the like of the components described in the present examples are not intended to limit the scope of this invention to them alone, unless otherwise stated.

### Device Configuration

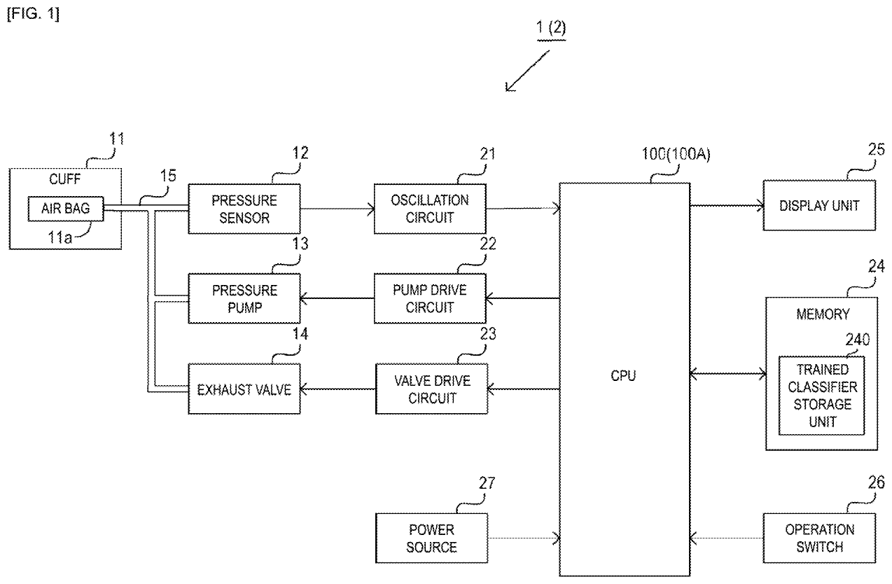

FIG. 1 is a schematic diagram illustrating a hardware configuration of a blood pressure measurement device 1 according to the present example.

The blood pressure measurement device 1 includes a cuff 11, a pressure sensor 12, a pressure pump 13, an exhaust valve 14, an air tube 15, an oscillation circuit 21, a pump drive circuit 22, a valve drive circuit 23, a display unit 25, a memory 24, an operation switch 26, a power source 27, and a CPU 100. The blood pressure measurement device 1 corresponds to a blood pressure measurement device of the present invention. The cuff 11 corresponds to a cuff of the present invention.

The cuff 11 includes an air bag 11a in which air is contained. The cuff 11 is provided with the pressure sensor 12 for detecting a pressure in the air bag 11a of the cuff 11 (hereinafter referred to as “cuff pressure”), the pressure pump 13 for supplying air to the air bag 11a, and the exhaust valve 14 that is opened and closed for maintaining the pressure in the air bag 11a or discharging the air in the air bag 11a, via the air tube 15.

Furthermore, the blood pressure measurement device 1 includes the central processing unit (CPU) 100 for controlling each unit of the device, the memory 24 for storing data such as a program and parameters executed for determining the wrapping strength, including a trained classifier for determining wrapping strength (described below), a program executed for blood pressure measurement processing, cuff pressure, pressurization rate, and blood pressure measurement results, the display unit 25 for displaying various types of information such as wrapping strength determination results and blood pressure measurement results, the operation switch 26 for inputting various instructions for measurement, and the power source 27 for supplying power to each unit of the device, such as the CPU.

In addition, the oscillation circuit 21 outputs, to the CPU 100, a signal having an oscillation frequency corresponding to an output value of the pressure sensor 12. The pump drive circuit 22 controls driving of the pressure pump 13, based on a control signal output from the CPU 100. The valve drive circuit 23 controls the opening and closing of the exhaust valve 14, based on a control signal output from the CPU 100.

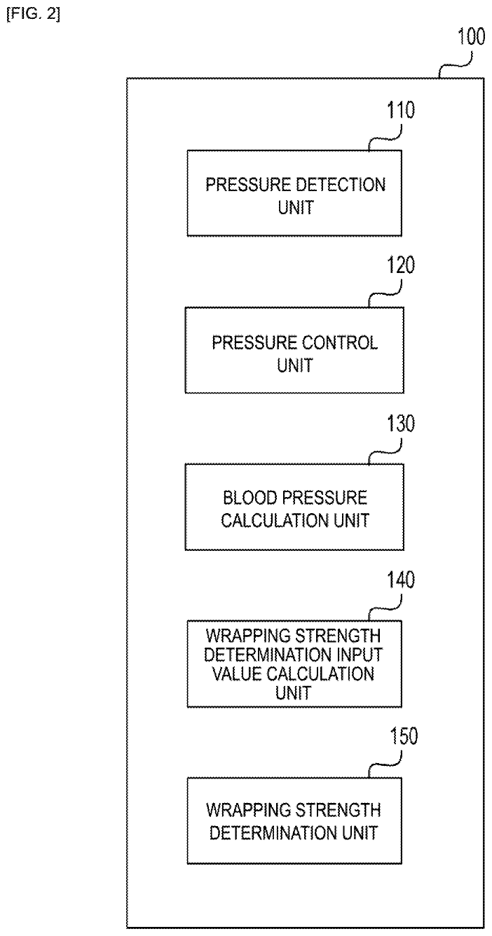

FIG. 2 is a functional block diagram of the blood pressure measurement device 1.

The CPU 100 includes a pressure detection unit 110, a pressure control unit 120, a blood pressure calculation unit 130, a wrapping strength determination input value calculation unit 140, and a wrapping strength determination unit 150. The pressure detection unit 110, the pressure control unit 120, the blood pressure calculation unit 130, and the wrapping strength determination unit 150 correspond to a pressure detection unit, a pressure control unit, a blood pressure calculation unit, and a wrapping strength determination unit of the present invention, respectively.

An output signal of the oscillation circuit 21 is input to the pressure detection unit 110. The pressure detection unit 110 detects an oscillation frequency of the input signal and converts the detected oscillation frequency into a pressure value signal. The pressure detection unit 110 includes an HPF (High Pass Filter) unit that extracts and outputs a pressure pulse wave signal by performing HPF processing on the pressure value signal, and an LPF (Low Pass Filter) unit that extracts and outputs a cuff pressure signal by performing LPF processing on the pressure value signal. The cuff pressure signal indicating the cuff pressure detected in a time-series manner from the LPF unit of the pressure detection unit 110 is stored in a predetermined area of the memory 24.

The pressure control unit 120 controls a cuff pressure of the cuff 11 by controlling operations of the pump drive circuit 22 and the valve drive circuit 23.

The blood pressure calculation unit 130 receives the pressure pulse wave signal extracted by the HPF unit of the pressure detection unit 110, and processes the received pressure pulse wave signal in accordance with an oscillometric method, thereby calculating a diastolic blood pressure (minimum blood pressure) and a systolic blood pressure (maximum blood pressure).

The wrapping strength determination input value calculation unit 140 receives a cuff pressure signal indicating the cuff pressure detected in a time-series manner from the LPF unit of the pressure detection unit 110. The wrapping strength determination input value calculation unit 140 calculates a wrapping strength determination input value to be input to a trained classifier used for wrapping strength determination, which will be described below, based on the cuff pressure signals that are input in a time-series manner. The wrapping strength determination input value will be described below.

The wrapping strength determination unit 150 reads a trained classifier for determining wrapping strength from a trained classifier storage unit 240 of the memory 24, and the wrapping strength determination input value calculated by the wrapping strength determination input value calculation unit 140 is input to this trained classifier for determining wrapping strength. The trained classifier for determining wrapping strength outputs any one of the wrapping strengths of loose wrapping, proper wrapping, and tight wrapping as a classification result in response to the input. Details on the method for determining the wrapping strength will be described below.

### Blood Pressure Measurement Method

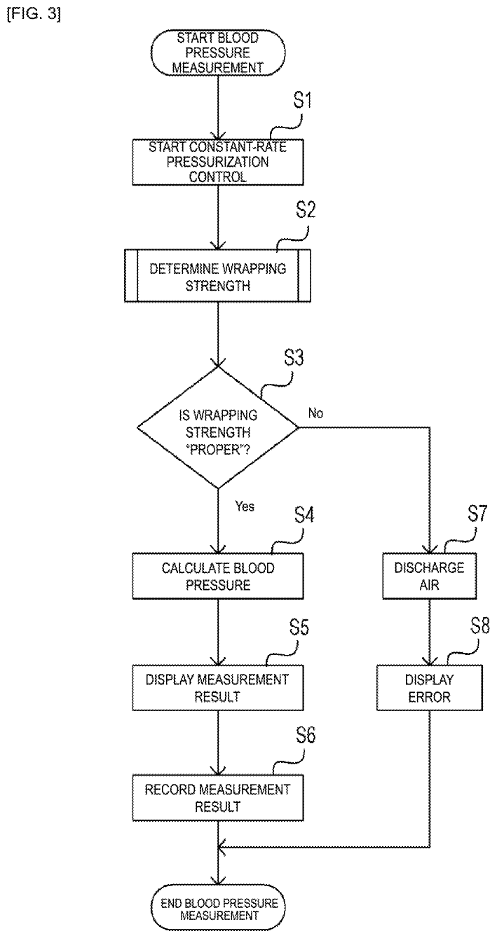

FIG. 3 is a flowchart illustrating an entire processing procedure of a blood pressure measurement method that is executed by the blood pressure measurement device 1. The entire processing for blood pressure measurement illustrated in FIG. 3 is stored in advance as a blood pressure measurement program in a predetermined area of the memory 24, and is implemented when the CPU 100 reads and executes the program from the memory 24. The program may also be stored in a computer-readable storage medium and read from the storage medium into the blood pressure measurement device 1.

When measuring blood pressure, the subject has a cuff 11 wrapped around a measurement target site in advance. The following example describes the measurement target site as the upper arm, but the measurement target site is not limited to the upper arm; it may also be the wrist or the like. In addition, the description will be made assuming that the subject performs a predetermined setting using the operation switch 26 and instructs the device to start blood pressure measurement. Note that, in the blood pressure measurement device 1 that has received the instruction to start blood pressure measurement, predetermined initialization such as opening the exhaust valve 14 and setting the cuff pressure to the atmospheric pressure (initial pressure) is executed.

When blood pressure measurement is started, the pressure control unit 120 starts constant-rate pressurization control (step S1). At this time, the pressure control unit 120 controls the pump drive circuit 22 so that the pressurization rate of the cuff pressure reaches a predetermined target value (referred to as “constant-rate pressurization control”). This constant-rate pressurization control can be performed, for example, by PID control, but the control method is not limited to this.

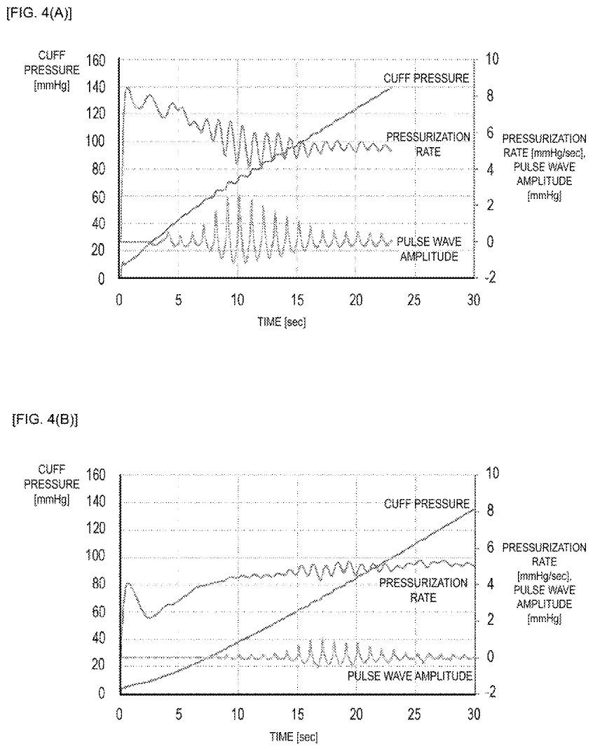

FIG. 4(A) illustrates time changes in cuff pressure, pressurization rate, and pulse wave amplitude under constant-rate pressurization control for a subject with an arm circumference of 22 cm, which is the circumference of the upper arm as a measurement target site. Furthermore, FIG. 4(B) illustrates time changes in cuff pressure, pressurization rate, and pulse wave amplitude under constant-rate pressurization control for a subject with an arm circumference of 36 cm.

As illustrated in FIG. 4(A) and FIG. 4(B), even when the same constant-rate pressurization control is performed, the time changes in cuff pressure and pressurization rate differ depending on the arm circumference of the upper arm of the subject to which the blood pressure measurement device 1 is attached.

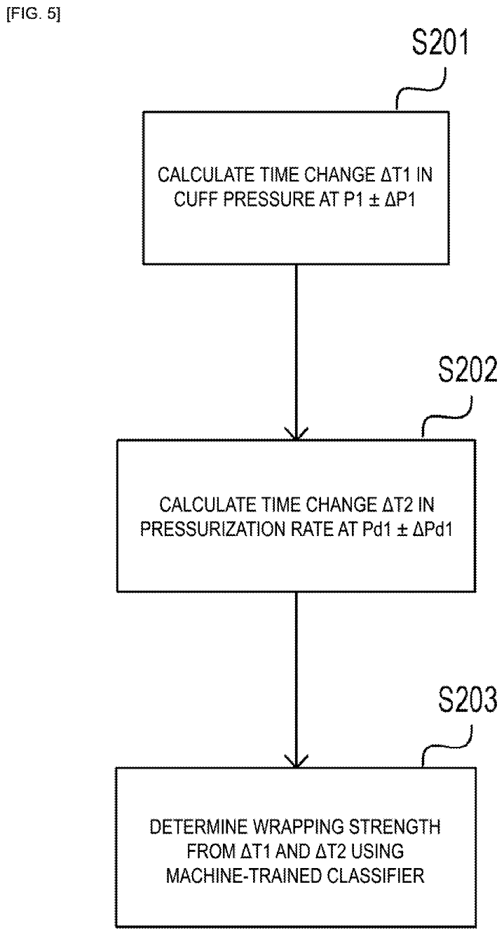

During the constant-rate pressurization control process, the wrapping strength determination unit 150 executes wrapping strength determination processing illustrated in FIG. 5, based on the determination input value calculated by the wrapping strength determination input value calculation unit 140 (step S3). Thus, the wrapping strength determination processing is performed under constant-rate pressurization control.

In step S3, the wrapping strength determination processing determines whether the wrapping strength of the cuff 11 falls into one of three categories: loose wrapping, proper wrapping, or tight wrapping. When the cuff 11 is wrapped around a measurement target site such as an upper arm to form a cylindrical shape, and when a circumferential length of the cylinder is substantially equal to a circumference of the measurement target site, a pressure applied to the measurement target site by the cuff 11 becomes an appropriate level for blood pressure measurement, and therefore such a state is referred to as proper wrapping. When the circumferential length of the cylinder formed by the cuff 11 is shorter than the circumference of the measurement target site, the cuff 11 is tightly wrapped around the measurement target site, and a pressure applied to the measurement target site by the cuff 11 is higher than the appropriate level, and therefore such a state is referred to as tight wrapping. When the circumferential length of the cylinder formed by the cuff 11 is longer than the circumference of the measurement target site, the cuff 11 is loosely wrapped around the measurement target site, and a pressure applied to the measurement target site by the cuff 11 is lower than the appropriate level, and therefore such a state is referred to as loose wrapping.

The wrapping strength determination processing will be described below with reference to FIG. 5.

First, the wrapping strength determination input value calculation unit 140 calculates a time change ΔT1 in cuff pressure at P1±ΔP1, that is, a time ΔT1 for the cuff pressure to change from P1−ΔP1 to P1+ΔP1, from the output from the LPF unit of the pressure detection unit 110 (which may also be data read from the memory 24) (step S201).

Next, the wrapping strength determination input value calculation unit 140 calculates a time change ΔT2 in pressurization rate at Pd1 ±ΔPd1, that is a time ΔT2,when Pd1 is the pressurization rate (the derivative of the cuff pressure) corresponding to the cuff pressure P1, for the pressurization rate to change from Pd1 −ΔPd1 to Pd1 +ΔPd1,from the output of the LPF unit of the pressure detection unit 110 (which may also be data read from the memory 24) (step S202).

Then, ΔT1 and ΔT2 calculated by the wrapping strength determination input value calculation unit 140 are input to the trained classifier for determining wrapping strength read from memory 24. This trained classifier for determining wrapping strength is a classifier generated by machine learning that, in response to inputs of changes in cuff pressure and pressurization rate, classifies and outputs the wrapping strength as either loose wrapping, proper wrapping, or tight wrapping. Based on the input ΔT1 and ΔT2, the trained classifier for determining wrapping strength determines the wrapping strength of the blood pressure measurement device 1 by classifying the wrapping strength by the subject (step S203).

This trained classifier for determining wrapping strength can be generated as follows. (1) First, as described above, ΔT showing the time change in cuff pressure, ΔT2 showing the time change in pressurization rate of the cuff pressure, and the wrapping strength of the cuff 11 at the time of measurement when such data was obtained are acquired. (2) A classification model is prepared that takes the time change ΔT1 in cuff pressure and the time change ΔT2 in pressurization rate of the cuff pressure as inputs and outputs the wrapping strength as either loose wrapping, proper wrapping, or tight wrapping. (3) The prepared classification model is caused to perform machine learning using the acquired data as learning data. As the classification model, an appropriate model can be employed. Furthermore, as a method of machine learning, an appropriate method such as deep learning can be employed. Such a classifier may be configured such that a learning device in an external system of the blood pressure measurement device 1 acquires, from a plurality of blood pressure measurement devices 1 via a network, ΔT showing the time change in cuff pressure, ΔT2 showing the time change in pressurization rate of the cuff pressure, and the wrapping strength of the cuff 11 at the time of measurement, and based on the data, acquires, via the network, a generated or updated trained classifier and stores the trained classifier in the memory 24 of the blood pressure measurement device 1, or may be provided in a form that allows the blood pressure measurement device 1 to import the trained classifier by a recording medium in which parameters and the like are stored.

Referring back to the flowchart in FIG. 3, the entire processing of blood pressure measurement will be continuously described.

In step S203, if it is determined that the wrapping strength is proper, the blood pressure calculation unit 130 acquires a pressure pulse wave for blood pressure measurement from the HPF unit of the pressure detection unit 110 and performs blood pressure calculation processing to calculate blood pressure values such as systolic blood pressure and diastolic blood pressure using the oscillometric method (step S4).

Here, following the end of the wrapping strength determination processing in step S2, the blood pressure calculation processing in step S4 is performed while maintaining constant-rate pressurization control. This eliminates noise generation due to control switching after the end of the wrapping strength determination processing, enabling the acquisition of a clean pressure pulse wave even at low pressure.

The CPU 100 displays the measurement result such as the blood pressure value calculated in step S4 on the display unit 25 of the blood pressure measurement device 1 (step S5). Then, the CPU 100 records the measurement result such as the blood pressure value calculated in step S4 in a predetermined area of the memory 24 of the blood pressure measurement device 1 (step S6), and ends the blood pressure measurement processing.

In the wrapping strength determination processing of step S2, if it is determined that the wrapping strength is not proper, that is, if it is determined to be loose wrapping or tight wrapping, the blood pressure calculation unit 130 does not perform the blood pressure calculation processing, but the pressure control unit 120 controls the valve drive circuit 23 to open the exhaust valve 14 and discharge air from the air bag 11a of the cuff 11 (step S7). Then, the CPU 100 displays an error on the display unit 25 (step S8) and ends the blood pressure measurement processing.

### Example 2

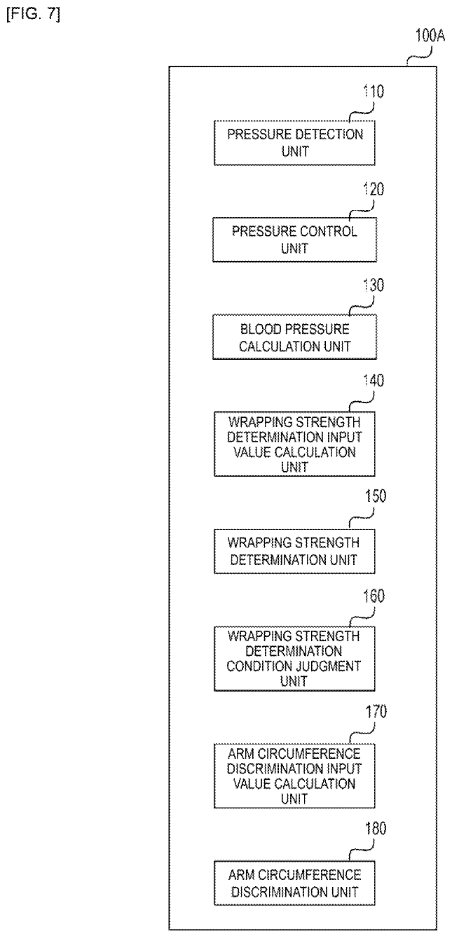

FIG. 7 is a functional block diagram of a blood pressure measurement device 2 according to Example 2. The configuration of the blood pressure measurement device 2, excluding a CPU 100A, is the same as that of the blood pressure measurement device 1 according to Example 1, and therefore a detailed description will be omitted. The blood pressure measurement device 2 has a function of discriminating the arm circumference of the upper arm to which the blood pressure measurement device 2 is attached, prior to the wrapping strength determination processing.

The CPU 100A includes, in addition to the pressure detection unit 110, the pressure control unit 120, the blood pressure calculation unit 130, the wrapping strength determination input value calculation unit 140, and the wrapping strength determination unit 150, a wrapping strength determination condition judgment unit 160, an arm circumference discrimination input value calculation unit 170, and an arm circumference discrimination unit 180.

The wrapping strength determination condition judgment unit 160 judges whether the conditions for determining the wrapping strength are satisfied. The conditions for determining the wrapping strength will be described below.

The arm circumference discrimination input value calculation unit 170 receives a cuff pressure signal indicating the cuff pressure detected in a time-series manner from the LPF unit of the pressure detection unit 110. The arm circumference discrimination input value calculation unit 170 calculates an arm circumference discrimination input value to be input to a trained classifier used for arm circumference discrimination, which will be described below, based on the cuff pressure signals that are input in a time-series manner. The arm circumference discrimination input value will be described below.

The arm circumference discrimination unit 180 reads a trained classifier for arm circumference discrimination from the trained classifier storage unit 240 of the memory 24, and the arm circumference discrimination input value calculated by the arm circumference discrimination input value calculation unit 170 is input to this trained classifier for arm circumference discrimination. The trained classifier for arm circumference discrimination discriminates the arm circumference in response to the input and outputs the result. Details on the method for arm circumference discrimination will be described below. Here, the arm circumference discrimination unit 180 corresponds to a circumference discrimination unit of the present invention.

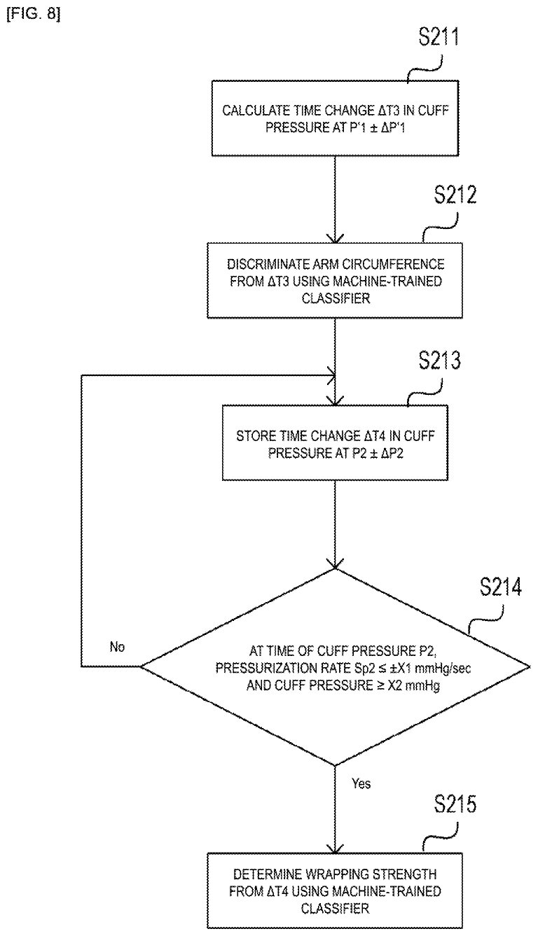

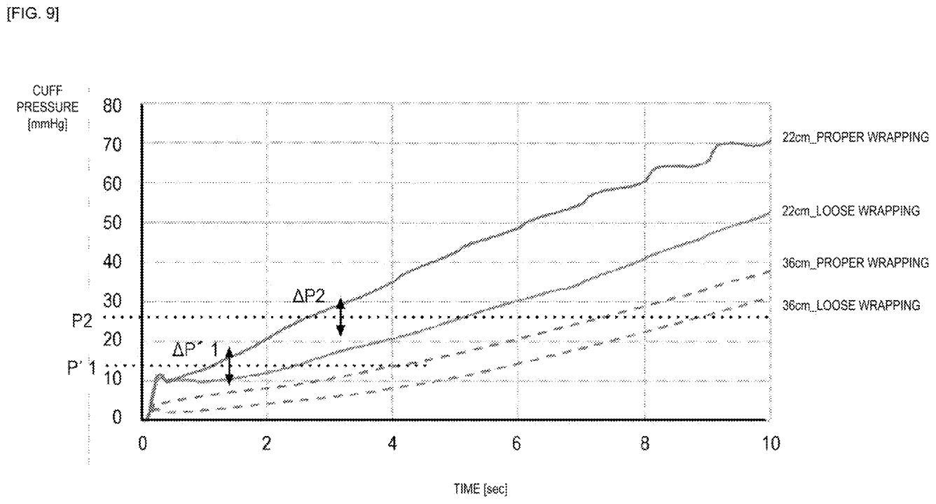

Referring to FIG. 8, the wrapping strength determination processing in the blood pressure measurement device according to Example 2 will be described. FIG. 9 is a graph illustrating a time change in cuff pressure for different arm circumferences and different wrapping strengths.

First, the arm circumference discrimination input value calculation unit 170 calculates a time change ΔT3 in cuff pressure at P′1±ΔP′1, that is, a time change ΔT3 for the cuff pressure to change from P′1−ΔP′1to P′1+ΔP′1, from the output from the LPF unit of the pressure detection unit 110 (which may also be data read from the memory 24) (step S211). Here, the cuff pressure P′ is set to a value corresponding to the initial rise in cuff pressure under constant-rate pressurization control. The cuff pressure P′1 may be the same as P1 described in Example 1.

Next, the ΔT3 calculated by the arm circumference discrimination input value calculation unit 170 is input to the trained classifier for arm circumference discrimination read from the trained classifier storage unit 240 of the memory 24. This trained classifier for arm circumference discrimination is a classifier generated by machine learning that discriminates and outputs the arm circumference in response to the input of the waveform change during the initial rise under constant-rate pressurization control. Based on the input ΔT3, the trained classifier for arm circumference discrimination discriminates the arm circumference of the subject to which the blood pressure measurement device 1 is attached (step S212). As a method of machine learning, an appropriate method such as deep learning can be employed.

Next, the wrapping strength determination input value calculation unit 140 calculates a time change ΔT4 in cuff pressure at P2±ΔP2, that is, a time change ΔT4 for the cuff pressure from P2−ΔP2 to P2+ΔP2, from the output from the LPF unit of the pressure detection unit 110 (which may also be data read from the memory 24) (step S213). The cuff pressure P2 may be set according to the arm circumference discriminated in step S212.

Then, the wrapping strength determination condition judgment unit 160 judges whether the wrapping strength determination condition is satisfied at the time of the cuff pressure P2, namely whether the pressurization rate Sp2≤±X1 mmHg/sec and the cuff pressure P2 is X2 mmHg or greater. The wrapping strength determination condition is substantially for judging whether the pressurization rate is stable and whether the necessary information for determining the wrapping strength has been obtained. X1 and X2 are set from this viewpoint and stored in the memory 24, and the wrapping strength determination condition judgment unit 160 reads them and executes a judgment. For example, X2 can be set to a value approximately twice that of P1. In this case, the wrapping strength determination condition judgment unit 160 corresponds to a determination condition judgment unit of the present invention.

If it is judged in step S214 that the wrapping strength determination condition is not satisfied, the process returns to step S213, and steps S213 and S214 are repeated for a new P2.

If it is judged in step S214 that the wrapping strength determination condition has been satisfied, the process proceeds to step S215. Here, ΔT4 calculated by the wrapping strength determination input value calculation unit 140 in step S213 is input to the trained classifier for determining wrapping strength read from the trained classifier storage unit 240 of the memory 24. This trained classifier for determining wrapping strength is a classifier generated by machine learning that, in response to input of changes in cuff pressure, classifies and outputs the wrapping strength as either loose wrapping, proper wrapping, or tight wrapping. Based on the input ΔT4, the trained classifier for determining wrapping strength determines the wrapping strength of the blood pressure measurement device 1 by classifying the wrapping strength by the subject. Here, a trained classifier for determining wrapping strength is used, which determines wrapping strength using the change in cuff pressure as input. However, a trained classifier for determining wrapping strength may also be used, which determines wrapping strength in response to the input to which the arm circumference discriminated in step S212 is added. A trained classifier for determining wrapping strength can be generated in the same manner as in Example 1.

The blood pressure measurement processing and the like after the end of the wrapping strength determination processing in the blood pressure measurement device 2 are the same as steps S4 to S8 described for the blood pressure measurement device 1 according to Example 1 with reference to the flowchart in FIG. 3.

In the present example as well, since the blood pressure calculation processing in step S4 is performed while maintaining constant-rate pressurization control immediately after the end of the wrapping strength determination processing in step S2, there is no noise generated due to control switching after the end of the wrapping strength determination processing, and a clean pressure pulse wave can be acquired even at low pressure.

In the present example, the arm circumference discrimination processing and the wrapping strength determination processing are executed for the cuff pressures P1′ and P2, respectively, but they may also be executed for a plurality of cuff pressures. Furthermore, the wrapping strength determination processing according to Example 1 may be employed as the wrapping strength determination processing.

### REFERENCE SIGNS LIST

1, 2 Blood pressure measurement device
11 Cuff
110 Pressure detection unit
120 Pressure control unit
130 Blood pressure calculation unit
150 Wrapping strength determination unit

1. A blood pressure measurement device comprising:
a cuff configured to be wrapped around a measurement target site;
a pressure detection unit configured to detect a cuff pressure inside the cuff;
a pressure control unit configured to control the cuff pressure;
a blood pressure calculation unit configured to perform blood pressure calculation processing of calculating a blood pressure of a subject, based on the detected cuff pressure; and
a wrapping strength determination unit configured to perform wrapping strength determination processing of determining a wrapping strength of the cuff around the measurement target site,
wherein the pressure control unit performs constant-rate pressurization control such that the cuff pressure is pressurized at a constant rate through the wrapping strength determination processing performed after a start of pressurization of the cuff pressure and the blood pressure calculation processing performed after an end of the wrapping strength determination processing.

2. The blood pressure measurement device according to claim 1, wherein the wrapping strength determination unit determines the wrapping strength in the wrapping strength determination processing, based on time changes in the cuff pressure and in a pressurization rate of the cuff pressure.

3. The blood pressure measurement device according to claim 2, wherein the wrapping strength determination unit determines the wrapping strength in the wrapping strength determination processing by using a trained classifier that classifies the wrapping strength, based on input time changes in the cuff pressure and in the pressurization rate of the cuff pressure.

4. The blood pressure measurement device according to claim 1, further comprising a determination condition judgment unit configured to judge whether a condition for starting the wrapping strength determination processing is satisfied.

5. The blood pressure measurement device according to claim 2, further comprising a determination condition judgment unit configured to judge whether a condition for starting the wrapping strength determination processing is satisfied.

6. The blood pressure measurement device according to claim 3, further comprising a determination condition judgment unit configured to judge whether a condition for starting the wrapping strength determination processing is satisfied.

7. The blood pressure measurement device according to claim 1, further comprising a circumference discrimination unit configured to discriminate a circumference of the measurement target site around which the cuff is to be wrapped.

8. The blood pressure measurement device according to claim 2, further comprising a circumference discrimination unit configured to discriminate a circumference of the measurement target site around which the cuff is to be wrapped.

9. The blood pressure measurement device according to claim 3, further comprising a circumference discrimination unit configured to discriminate a circumference of the measurement target site around which the cuff is to be wrapped.

10. The blood pressure measurement device according to claim 1, wherein the blood pressure calculation unit judges whether to perform the blood pressure calculation processing according to a result of the wrapping strength determination processing.

11. The blood pressure measurement device according to claim 2, wherein the blood pressure calculation unit judges whether to perform the blood pressure calculation processing according to a result of the wrapping strength determination processing.

12. The blood pressure measurement device according to claim 3, wherein the blood pressure calculation unit judges whether to perform the blood pressure calculation processing according to a result of the wrapping strength determination processing.

13. The blood pressure measurement device according to claim 1, wherein a target value of a pressurization rate of the cuff pressure in the constant-rate pressurization control during the wrapping strength determination processing is equal to a pressurization rate of the cuff pressure in the constant-rate pressurization control during the blood pressure calculation processing.

14. The blood pressure measurement device according to claim 2, wherein a target value of a pressurization rate of the cuff pressure in the constant-rate pressurization control during the wrapping strength determination processing is equal to a pressurization rate of the cuff pressure in the constant-rate pressurization control during the blood pressure calculation processing.

15. The blood pressure measurement device according to claim 3, wherein a target value of a pressurization rate of the cuff pressure in the constant-rate pressurization control during the wrapping strength determination processing is equal to a pressurization rate of the cuff pressure in the constant-rate pressurization control during the blood pressure calculation processing.

14. A blood pressure measurement method comprising:
detecting a cuff pressure inside a cuff configured to be wrapped around a measurement target site;
performing constant-rate pressurization control of maintaining a pressurization rate of the cuff pressure at a constant target value;
performing wrapping strength determination processing of determining a wrapping strength around the measurement target site during the constant-rate pressurization control; and
performing blood pressure calculation processing of calculating a blood pressure of a subject, based on the cuff pressure during the constant-rate pressurization control.

15. A blood pressure measurement recording medium for causing a computer to execute:
detecting a cuff pressure inside a cuff configured to be wrapped around a measurement target site;
performing constant-rate pressurization control of maintaining a pressurization rate of the cuff pressure at a constant target value;
performing wrapping strength determination processing of determining a wrapping strength around the measurement target site during the constant-rate pressurization control; and
performing blood pressure calculation processing of calculating a blood pressure of a subject, based on the cuff pressure during the constant-rate pressurization control.

16. A method for generating a classifier used in wrapping strength determination processing in a blood pressure measurement recording medium for causing a computer to execute:
detecting a cuff pressure inside a cuff configured to be wrapped around a measurement target site;
performing constant-rate pressurization control of maintaining a pressurization rate of the cuff pressure at a constant target value;
performing the wrapping strength determination processing of determining a wrapping strength around the measurement target site during the constant-rate pressurization control; and
performing blood pressure calculation processing of calculating a blood pressure of a subject, based on the cuff pressure during the constant-rate pressurization control, the method comprising:
machine-learning a classifier configured to output the wrapping strength around the measurement target site as a classification result in response to input of time changes in the cuff pressure and the pressurization rate of the cuff pressure by using a time change in the cuff pressure, a time change in the pressurization rate of the cuff pressure, and the wrapping strength around the measurement target site as learning data; and
generating the trained classifier.
